(19) United States
(12) Patent Application Publication
(10) Pub. No.: US 2002/0004042 A1
(43) Pub. Date: Jan. 10, 2002

(54) Gene therapy for pulmonary edema using adenovirus vectors encoding a beta2adrenergic receptor gene

(76) Inventor: Phillip H. Factor, Glencoe, IL (US)

Correspondence Address: BARNES & THORNBURG, 2600 CHASE PLAZA, 10 LASALLE STREET, CHICAGO, IL, 60603

(21) Appl. No.: 09/835,817

(22) Filed: Apr. 16, 2001

Related U.S. Application Data
(60) Provisional application No. 60/228,254, filed on Apr. 18, 2000.

(51) Int. Cl. A61K 48/00; C12N 15/861

(52) U.S. Cl. 424/93.21; 514/44; 435/320.1

(57) ABSTRACT

Methods and compositions are provided for gene therapy for pulmonary edema by the transfer of a human β₂AR gene to lung epithelial cells for the purpose of improving responsiveness to endogenous catecholamines and increasing active Na⁺ transport and pulmonary edema clearance in vivo. Recombinant adenoviral vectors mediate the transfer of the β₂AR gene into lung epithelial cells. The vectors employ expression control sequences consisting of viral derived promoter elements linked to cDNAs that express a human β₂AR genes. This gene has been shown to be capable of augmenting the function of transport proteins that generate the transepithelial osmotic gradient responsible for the movement of water across epithelial membranes to increase pulmonary edema clearance in mammalian lungs.

This application claims priority from co-pending U.S. provisional application Ser. No. 60/228,254 filed Apr. 18, 2000.

### BACKGROUND OF THE INVENTION

Methods and compositions are provided for gene therapy for pulmonary edema by the transfer of a β₂ Adrenergic Receptor (β₂AR) gene to lung epithelial cells for the purpose of increasing responsiveness to endogenous catecholamines and improving pulmonary edema clearance in vivo.

Cardiogenic and non-cardiogenic pulmonary edema affect millions of people each year causing substantial morbidity and mortality (Consortium, 1997). The alveoli of these people flood with liquid from pulmonary capillaries which compromises oxygen transfer to the systemic circulation (Hall and Wood, 1986). This sequence of events results in hypoxemia, hypercapnea, and death if no corrective measures are taken.

Unfortunately, no specific treatment for pulmonary edema is available. Current therapy is entirely supportive and includes diuretic therapy to reduce pulmonary capillary hydrostatic pressure. This therapy has been shown to reduce edema accumulation but does not influence pulmonary edema clearance (Sznajder et al., 1986). In many cases, this therapy leads to inappropriately low left ventricular end diastolic volumes, reduced cardiac output, hypotension, and decreased peripheral oxygen delivery (Hall and Wood, 1986). Therapies that improve or reconstitute the lung's ability to keep itself dry could reduce the morbidity associated with pulmonary edema.

Edema accumulates in the alveolus of the lung as a result of increases in capillary permeability and/or hydrostatic pressure, as described by the well known Starling's equation (Staub, 1974). Conversely, edema is cleared from the alveolus as a result of active transport of Na⁺ out from the alveolar air space. This Na⁺ transport is due to the coordinated action of Na,K-ATPases that are located on the basolateral surface of alveolar type 2 epithelial cells (AT2) and Na⁺ channels, which are found on the apical surface of these cells. These transport proteins generate a transepithelial osmotic gradient that causes fluid movement out of the alveolar airspace via trans- and para-cellular pathways. The expression and function of these molecules is reduced in some forms of pulmonary edema. Methods and compositions to increase alveolar active Na⁺ transport are thus needed to improve these conditions.

Lung liquid (pulmonary edema) clearance resulting from active Na⁺ transport was reported in live animal models, in isolated rat lungs, and in humans (Effros et al., 1989; Goodman et al., 1983; Matthay and Wiener-Kronish, 1990). Supporting the role of active Na⁺ transport in lung liquid clearance are experiments in isolated rat lungs which demonstrate that lung liquid clearance is completely stopped by hypothermia (via inhibition of active transport), and is decreased by both amiloride (a Na⁺ channel inhibitor) and ouabain (a Na,K-ATPase inhibitor).

It has been observed that β-adrenergic agonists increase Na,K-ATPase and Na⁺ channel function isolated rat AT2 cells in vitro, rat, sheep, dog lungs in vivo, and in human lung explants (Berthiaume et al., 1987, 1988; Bertorello et al., 1999; Goodman et al., 1984; Minakata et al., 1998; Sakuma et al., 1997). Correlative in vivo studies from rat, mouse, dog, and sheep models indicate that β-agonists increase alveolar active Na⁺ transport and edema clearance. Saldias, et al. reported that the β-adrenergic agonist isoproterenol increases lung liquid clearance in isolated rat lungs by >100% by increasing recruitment of Na,K-ATPases to the cell membrane (Saldias et al., 1998). Similar results from rat AT2 cells have also been reported (Bertorello, et al., 1999). These studies suggest that β-adrenergic agonists affect lung liquid (e.g. pulmonary edema) clearance via modulation of alveolar epithelial Na,K-ATPases.

Autoradiographic studies of rat lungs were interpreted to show uniform distribution of β receptors in the alveolar wall suggesting that they are present in both type 1 and 2 alveolar epithelial cells (Carstairs et al., 1985; Nishikawa et al., 1993; 1994). Tibayan treated rat lungs with dopamine (aβ₁ agonist) and dobutamine (a non-specific β-agonist) and concluded that β-adrenergic agonists affect clearance via β₂-adenergic receptors (Tibayan et al., 1997). Studies of the effects of catecholamines on alveolar liquid clearance in normal mice, rat models of sepsis and neurogenic pulmonary edema, and adrenalectomized sheep have similarly reported that β-adrenergic responsive alveolar liquid clearance is mediated via β₂ receptors (Campbell et al., 1999; Icard and Saumon, 1999; Lane et al., 1998; Pittet et al., 1994).

Endogenous catecholamines were reported to contribute to alveolar liquid clearance in septic rats (Pittet, et al., 1994). These authors proposed that catecholamine responsive alveolar liquid clearance is an important, protective mechanism that helps keep the lung dry during pathological conditions. A recent report indicates that hemorrhagic shock causes an oxidant-mediated lung injury that eliminates responsiveness to exogenous and endogenous catecholamines (Modelska et al., 1999). This study suggests that in some forms of acute lung injury adrenergic responsive liquid clearance may be impaired. Additionally, the β-agonists terbutaline and isoproterenol were reported to improve lung liquid clearance in the setting of hyperoxic lung injury (Lasnier, et al., 1996; Saldias et al., 1999). β₂AR polymorphisms were reported and proposed to effect responses to β₂-agonists in humans (Liggett, 1997; Martinez et al., 1997). Whether these polymorphisms are important in lung liquid clearance remains to be determined.

The studies summarized above suggest that β-agonists affect alveolar solute transport via cAMP, cAMP-dependent kinases, PKC and other as yet undefined pathways that either increase function in, recruitment to, or stability of transport proteins in the cell membrane that lead to increased alveolar epithelial active Na⁺ transport.

Barnes, et al. reported that prolonged infusion of norepinephrine in guinea pigs is associated with greater reduction in β₂-receptor density and mRNA in the alveolar wall than in the airway (Nishikawa, et al., 1994). These observations suggest that β₂AR downregulation occurs in the alveolar epithelium and that prolonged exposure to β-agonists results in degradation of existing receptors, cessation of new synthesis, and/or destabilization of β₂AR message. These studies suggest that receptor overexpression can overcome endogenous regulatory mechanisms and potentiate β-adrenergic signaling by maintaining β₂AR receptors in the cell membrane.

Replication deficient adenoviruses are useful for gene transfer studies. They are tropic for the respiratory epithelium, infect non-replicating cells with high efficiency, and do not integrate into the host genome. The absence of a crucial gene (E₁ₐ) makes it impossible for them to replicate outside of cells expressing E₁ₐ. Hence, they do not propagate following infection of eukaryotic cells. These recombinant vehicles can be constructed with powerful promoters that allow high level, transient expression of a gene of interest. As such they are excellent vectors for the delivery and short-term expression of many genes. These vectors are also capable of gene transfer to cells in vitro. As such, these vectors are useful for the production of recombinant proteins.

Currently available treatments for pulmonary edema are unable to affect the lung's ability to remove excess fluid from the alveolar airspace without affecting other organs. Thus, no specific treatments for pulmonary edema are currently available.

### SUMMARY OF THE INVENTION

The invention relates methods and compositions for reducing pulmonary edema in acquired diseases of the mammalian lung. An aspect of the invention is to use adenoviruses to develop compositions for gene therapy for lung illnesses, including pulmonary edema. The method includes the following steps:

(a) obtaining a recombinant genetic vector including

(i) an adenovirus that has no nucleotide sequences encoding adenovirus proteins; and

(ii) nucleotide sequences encoding a human β₂ Adrenergic Receptor gene at levels that are an overexpression compared to levels in lung cells not having the genetic vector and

(b) transferring the genetic vector into epithelial cells of the lung under conditions that allow expression of the β₂AR gene.

The invention also relates a recombinant genetic vector including:

(a) an adenovirus that has no nucleotide sequences encoding adenovirus proteins; and

(b) nucleotide sequences of aβ₂ Adrenergic Receptor encoding a gene that expresses at levels that are an overexpression compared to levels in lung cells not having the genetic vector.

An aspect of the invention is a host cell into which a recombinant genetic vector has been transferred, said vector including:

(a) an adenovirus that has no nucleotide sequences encoding adenovirus proteins; and

(b) nucleotide sequences encoding a β₂ Adrenergic Receptor gene at levels that are an overexpression compared to levels in lung cells not having the genetic vector.

This invention provides methods and compositions for the transfer of a β₂ Adrenergic Receptor gene to lung epithelial cells for the purpose of increasing in vivo β₂ Adrenergic Receptor activity and improving lung liquid clearance. Preferred vectors for the treatment of acquired, acute/short-term illnesses of the lung are replication deficient adenovirus. These methods and compositions are designed to augment endogenous alveolar transport processes for the purposes of gene therapy for pulmonary edema.

### BRIEF DESCRIPTION OF THE DRAWINGS

FIG. 1 shows transgene expression assayed via northern (upper blot) and western (lower blot) analysis, expression was in human lung epithelial cells (A549) that were infected with 50 pfu/cell of a replication deficient first generation adenovirus that expressed a human β₂ adrenergic receptor cDNA (adβ₂AR) for 48 hours.

FIG. 2 shows expression of mouse fibroblasts (3T3) infected with 50 pfu/cell of a replication deficient first generation adenovirus that expresses a human β₂ adrenergic receptor cDNA (adβ₂AR); expression was for 48 hours prior to immunostaining with an anti-human β₂AR antibody.

FIG. 3 illustrates cAMP levels measured in human lung epithelial cells (A549) infected with 50 pfu/cell of adβ₂AR for 48 hours prior to treatment with procaterol (10⁻⁶ M×1 minute) and/or the β₂AR specific antagonist ICI 118,551 to test whether overexpression could increase β₂AR function in these cells; experimental vs. untreated controls (Student's t-test, *p<0.002).

FIG. 4 illustrates the effect of receptor overexpression on β₂-agonist responsive (procaterol) active Na⁺ transport in human lung epithelial cells (A549) infected with 50 pfu/cell of adβ₂AR for 48 hours; experimental vs. untreated, sham infected controls, *p<0.01; experimental vs. all other groups (Student's t-test !p<0.002).

FIG. 5 presents results of Western analysis (blots) of basolateral membrane fractions harvested from A549 cells infected with 50 pfu/cell of adβ₂AR for 48 hours prior to treatment with procaterol (10⁻⁶ M×10 minutes).

FIG. 6 shows a Western blot of lungs of normal adult male Sprague-Dawley rats infected with 4×10⁹ pfu of adβ₂AR for 7 days prior to Western analysis of whole lung tissue for the presence of a human β₂AR; virus was delivered to lungs using a surfactant based vehicle.

FIG. 7 shows lung liquid clearance measured in rat lungs infected with 4×10⁹ pfu of adβ₂AR 7 days prior to study; clearance was measured in the absence of β-agonists using a fluid-filled, isolated, perfused lung preparation; experimental vs. sham infected, *p<0.002.

FIG. 8 shows lung liquid clearance in adrenalectomized rats infected with adβ₂AR (4×10⁹ pfu×7 days) measured using an isolated, fluid-filled lung preparation.

FIG. 9 shows lung liquid clearance of normal adult male Sprague-Dawley rats infected with adβ₂AR or adNull for 7 days. Beginning 2 days after infection, they were treated with propranolol (1 mg/kg every 8 hours by gavage).

FIG. 10 shows results of tests to determine whether β-agonist induced receptor desensitization affects alveolar active Na⁺ transport; rats were treated with a β₂AR specific agonist (procaterol, 100 μg q 8 hrs by gavage) for 4-24 hours prior to measurement of alveolar liquid clearance and apical membrane receptor expression; control lungs were exposed to procaterol only during clearance measurements. Catecholamine responsive alveolar liquid clearance (see the graph on the left) was measured by treating isolated lungs with procaterol; β₂AR number was quantified by Western blot analysis (on the right) of apical membrane fractions of peripheral lung harvested from these same rats; experimental vs. untreated controls, *p<0.001 .

FIG. 11 illustrates the effect of β₂AR overexpression on receptor desensitization and downregulation; cAMP was measured in A549 cells infected with 50 pfu/cell of ad ₂AR or adNull for 48 hours prior to exposure to procaterol (10⁻⁸ M) for up to 120 minutes; cAMP levels (graph A), ouabain sensitive ⁸⁶Rb⁺ uptake (graph B) receptor number (graph C), receptor affinity for ligand (graph D).

FIG. 12 shows results of Western analysis of rtPCR (upper blot) of rat lungs infected with 1×10⁹ to 1×10¹⁰ pfu of a high-capacity, helpervirus-dependent adenovirus (hdβ₂AR) that expresses a human β₂AR cDNA for 72 hours; Western analysis (lower blot) of these rat lungs shows dose-dependent expression of a human β₂AR protein.

FIG. 13 shows photomicrographs of rat lungs infected with 5×10⁹ or 1×10¹⁰ pfu of hdβ₂AR for 72 hours; lung samples were harvested, fixed in formalin, imbedded in paraffin and sectioned for histologic evaluation.

FIG. 14 presents an evaluation of transgene function following hdβ₂AR infection of A549 cells for 48 hours prior to treatment with procaterol (10⁻⁶ M×10 minutes); experimental vs. control, *p<0.0001.

FIG. 15 shows lung liquid clearance in rats infected with hdβ₂AR, hdNull (a high-capacity, helper-virus dependent adenovirus that contains no cDNA) for 48 hours prior to measurement of clearance using a fluid-filled, perfused isolated lung preparation. Experimental vs. sham infected controls, *p<0.001.

FIG. 16 is a map of the expression cassette of hdβ₂AR.

FIG. 17 shows the sequence of the human β₂AR expression cassette; this sequence includes a human CMV immediate/early promoter (bp 1-587), a human β₂AR cDNA (bp 714-1955), and a bovine growth hormone polyadenylation signal (bp 1956-2225)

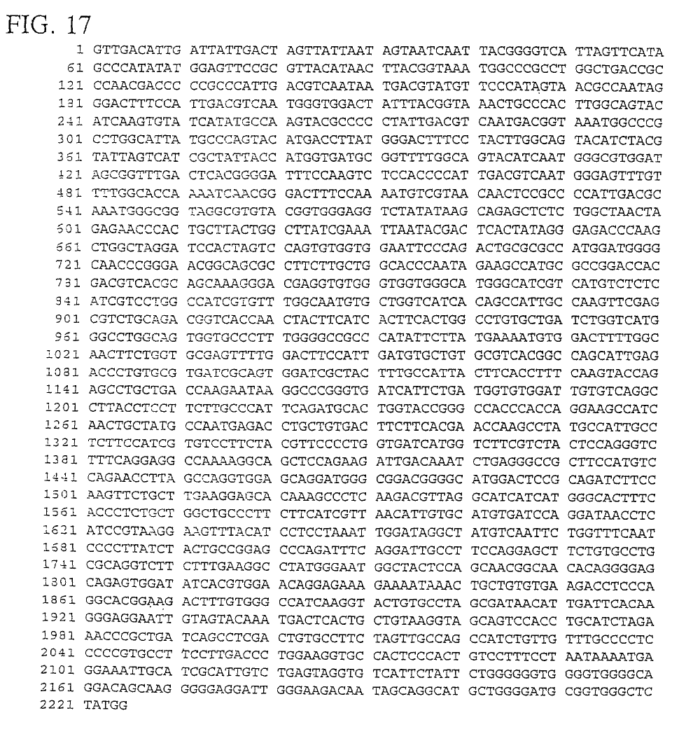

### DETAILED DESCRIPTION OF THE INVENTION

This invention provides therapeutic methods for the treatment of pulmonary edema (in animals and humans), and compositions to effect the methods. The methods involve introducing recombinant genes (DNA nucleotide sequences encoding a β₂AR) into alveolar epithelial cells in vivo. These cells transcribe and translate the recombinant genes thereby increasing expression and function of key transmembrane transport molecules (Na,K-ATPase and epithelial Na⁺ channels (ENaC)), thereby enhancing the lung's capacity to clear liquid from the alveolar airspace.

The methods of this invention involve the use of recombinant human adenoviral vectors for mediating the introduction of genes into alveolar epithelial cells. The recombinant adenoviral vectors of this invention produce superior results to other non-viral vectors (e.g. cationic lipids) in the transfer of genes into epithelial cells (Brody and Crystal, 1994). In particular, the methods of this invention involve the use of recombinant adenoviral vectors that mediate the transfer of β₂AR genes into alveolar epithelial cells.

The vectors described employ expression control sequences consisting of viral derived promoter elements linked to a cDNA that expresses a human β₂AR. This protein increases the transcription, recruitment to the cell membrane and activity of the key proteins (Na,K-ATPase and ENaC) responsible for generating the transepithelial osmotic gradient responsible for the movement of water across epithelial membranes. The transgenic protein (β₂AR) produced by this vector is a transmembrane molecule that stimulates the active solute transport necessary to effect alveolar edema clearance. Extensive testing of a 1^{st} generation β₂AR expressing adenovirus indicates that receptor overexpression markedly attenuates agonist induced receptor desensitization and downregulation.

The transepithelial movement of Na⁺ is dependent on the function of transport molecules on both the apical and basal cell membranes. The apical Na⁺ channel is a passive conduit for the entry of Na⁺ that opens to allow Na⁺ into the cell in response to changes in cAMP levels following activation of β₂AR. It is a tightly regulated channel that opens and closes in response to phosphorylation and dephosphorylation by membrane bound heterotrimetric G-proteins. It is considered by some to be the rate-limiting regulatory element in controlling vectorial Na⁺ transport. The basally located Na,K-ATPase uses high-energy phosphates to exchange intracellular Na⁺ for extracellular K⁺ creating a electrochemical gradient that drives transcellular Na⁺ and H₂O movement. Both the apical Na⁺ channel and Na,K-ATPase can be positively regulated by cAMP.

A high-capacity, helper-dependent adenoviral vector system of the present invention was selected to diminish adenoviral-induced host responses. These inflammatory responses have been attributed to the expression of adenoviral proteins in infected cells leading to a cytotoxic T cell response and clearance of infected cells (Brody et al., 1994; van Ginkel et al., 1997). The elimination of all adenoviral protein coding genes is expected to significantly abrogate this inflammatory response limiting host-mediated removal of adenovirus infected cells leading to more prolonged transgene expression than previously observed with first and second generation adenoviral vectors (Morral et al., 1999). This has been confirmed in recent publications that used these vectors to overexpress α₁ antitrypsin and leptin genes in mice (Morral et al., 1998; Morsy et al., 1998). As opposed to mice given first generation adenoviruses, the livers of mice given high-capacity, helper-dependent viruses were histologically “indistinguishable from (uninfected) control” and no elevation of hepatocellular enzyme levels were observed. This report indicated that a small quantity of helper adenovirus was concomitantly administered. These few first generation E1a⁻ vectors did not induce a significant host response. It was also shown that diminution of inflammation attenuates the loss of adenoviral transduced cells extending the duration of transgene expression. The helper-virus and cells (293Cre4) necessary to produce these helper-virus dependent vectors have been obtained from Merck by a material transfer agreement. The DNA vectors (helper-virus plasmids) containing a human β₂AR cDNA and promoter elements have been constructed by the inventors using human intronic DNA and fragments of wild-type human adenovirus type 5. These pieces were used to construct β₂AR expressing helper-virus dependent adenovectors.

Adenoviral receptors are located on the basolateral aspect of bronchial epithelial cells limiting gene transfer to airway cells (Walters et al., 1999). These receptors are likely expressed on the apical surface of alveolar epithelial cells (Factor et al., 1998a). Thus adenoviral vectors are uniquely suited for the goal of alveolar (as opposed to airway) gene transfer. Other pulmonary gene transfer strategies, while efficient, do not at this time provide the combination of high gene transfer efficiency, ease of production, and relative specificity for the alveolar epithelium. Due to this unique combination of attributes, recombinant adenoviruses are preferred vectors for gene therapy for pulmonary edema.

Replication deficient adenoviruses have been previously used for human gene transfer studies. However, most of these are phase I studies that have focused on the treatment of heritable conditions and cancer, and have yielded limited results. Gene therapy is not reported to treat acute or life threatening conditions. The use of these vectors for acquired conditions such as pulmonary edema represents a new use for these vectors.

This invention contemplates the use of constitutive promoter elements that include the immediate early promoter from human cytomegalovirus. This viral promoter element may be linked to expression control sequences that include the cDNAs for human, and other species, β₂AR genes. Additional regulatory sequences include the human SV40 t intron or growth hormone polyadenylation signals and other transcriptional control signals such as splice donor-acceptor sites.

The invention described herein employs the use of helper-virus dependent adenoviral vectors that are devoid of all wild-type protein encoding adenoviral genes. These vectors are produced by cloning of an expression cassette that includes a constitutive or inducible promoter that is linked to a human β₂AR cDNA followed by a polyadenylation sequence (e.g. SV40 t intron). This expression cassette is then inserted into a shuttle vector that contains the human adenovirus type 5 left and right inverted terminal repeats (ITR). Adjacent to the 3′ end of the left ITR is the packaging signal from human adenovirus type 5. In between the ITRs is 10-12 kb of intronic DNA from the human hypoxanthine guanine phosphoribosyl transferase (hGPRT) gene obtained from the American Tissue Type Collection (Stout and Caskey, 1985). An expression cassette containing a full-length human β₂AR cDNA is inserted within the human intronic DNA of the shuttle vector. When excised from its backbone by restriction endonuclease digestion, the 5′ end of the shuttle vector contains an adenoviral wild-type inverted terminal repeat and an adenoviral packaging signal. The 3′ end of this vector consists of an adenoviral wild-type inverted terminal repeat. The β₂AR cDNA containing shuttle vector (pHV ₂AR) is co-transfected into HEK293cre4 cells with a recombinant helper adenovirus, adLC8cluc (Parks et al., 1996). The adenovirus contains 2 loxP sites that flank the packaging signal. The remainder of adLC8cluc is similar to previously described first generation E1a⁻ deleted recombinant adenoviral vectors. When transfected into cells that express Cre recombinase (HEK293cre4, (Parks, et al., 1996)) the packaging signal is effectively excised making it impossible for newly synthesized adLC8cluc to be packaged into adenoviral capsids. The remainder of the adLC8cluc genome contains the protein encoding adenoviral genes necessary to allow rescue of DNA sequences from the β₂AR containing shuttle vector, pHVβ₂AR into a replication-incompetent, high-capacity, helper-virus dependent adenoviral vector. The adenovector thus produced is hdβ₂AR. This helper-virus dependent adenovirus is propagated by infecting confluent 15 cm tissue culture plates of HEK293 cells are infected with 3 pfu/cell. Following development of cytopathologic effect (CPE) the cells are harvested, concentrated and thermally disrupted via 6 cycles of freezing and thawing. The resultant cell lysate is cleared of cellular debris by high-speed centrifugation prior to purification through serial CsCl density gradient ultra-centrifugations. The resultant virus is dialyzed against 10 mM Tris HCl pH 7.4/1 mM MgCl/10% glycerol to remove CsCl prior to storage in 10% glycerol at −70° C. (McGrory et al., 1988). Helper-virus dependent adenovectors are titered based plaque production in HEK293cre4 cells co-infected with adLC8cluc and via optical density (1OD₂₆₀=1.1×10¹² viral particles). The presence of the expected cDNAs is confirmed by PCR. The presence of adLC8cluc is assayed by plaque production counts following infection of HEK293 cells grown under agarose and by measurement of luminescence in cell lysates in a luminometer. Wild-type adenovirus is assayed by plaque production counts in A549 cells and by PCR for E1a DNA sequences.

There are many forms of catecholamine receptors, so selecting a suitable one was part of the criteria for success of the invention. In the absence of prior data that showed a physiologically relevant affect of agonist induced β₂AR downregulation in the alveolar epithelium, it was unexpected that adenoviral-mediated β₂AR overexpression could increase lung liquid clearance.

Delivery strategies capable of widespread delivery of adenoviral vectors of the present infection to the alveolar airspace include the use of a functional surfactant based vehicle and methodologies for endotracheal instillation of adenovirus that include end-expiratory thoracic compression to drive end-expiratory lung volume toward residual volume and cause supra-physiologic inspiration to cause widespread distribution of vehicle and virus (Factor, et al., 1998a; Katkin et al., 1997). These methods include safe endotracheal intubations and use of a thoracic compression strategy aimed at driving end- expiratory lung volumes toward residual volume. These methods cause animals to take a deep/forceful inhalation that leads to widespread dissemination of adenovirus to all segments of the lung after thoracic compression is relinquished. This development is a factor allowing human gene therapy using high-capacity, helpervirus-dependent adenoviral vectors.

### EXAMPLES

The following examples present embodiments of the methods and compositions of the present invention.

### Example 1

### Adenoviral-mediated β₂ gene transfer to human alveolar epithelial cells in vitro.

First generation human type 5 replication deficient adenoviruses were deleted of sequences spanning all or part of the E1a, and E1b genes impairing the ability of these recombinant viruses to replicate outside of cells expressing these missing adenoviral DNA sequences. In the vector adβ₂AR, the early immediate promoter/enhancer element from human cytomegalovirus was used to drive transcription of a human β₂AR gene with an SV40 t intron polyadenylation signal downstream from the cDNA. An otherwise identical adenovirus containing no cDNA (adNull) was also used in this and the following examples.

These viruses were produced using techniques known to those skilled in the art that include use of a plasmid that contains an expression cassette that includes a cDNA encoding the a human β₂AR gene flanked by portions of the left end of a human type adenovirus genome (shuttle vector) and a plasmid that contains an adenovirus genome that exceeds the packaging limits of the adenoviral capsid. These plasmids were co-transfected into human embryonic kidney cells (HEK294). Homologous recombination between these two plasmids produces an adenovirus genome of appropriate size to fit into the adenoviral capsid. Homologous recombination is detected by the presence of typical cytopathologic effects in HEK293 cells. High titer recombinant adenoviruses were prepared by amplification in HEK293 cells using (Factor, et al., 1998a; Factor, et al., 1998b; McGrory, et al., 1988). Virus was purified from cell lysate by serial cesium chloride ultracentrifugation followed by desalting by dialysis against an isotonic, physiologic buffer. Virus thus produced was titered by enumeration of plaques produced following infection of sub-confluent HEK293 cells. Wild-type adenovirus contamination was assayed by plaque-production counts produced by serial dilution of adenovirus on A549 cells and via PCR for E1a DNA sequences. Presence of the desired cDNA was reconfirmed by PCR.

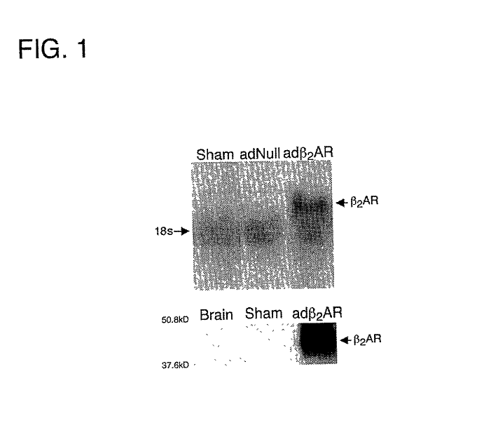

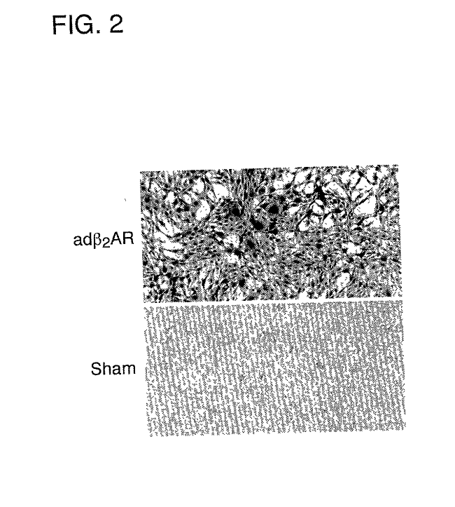

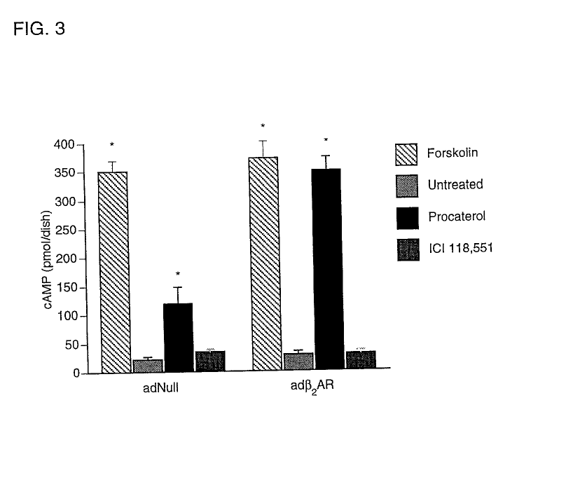

A human lung epithelial cell line (A549) was used for this example. A549 cells were plated on tissue culture treated plasticware and allowed to adhere for 24 hours prior to infection with adenovirus. A549 cells were infected with 50 pfu/cell of adβ₂AR and were contrasted to A549 cells infected with similar titers of adNull and to sham infected cells. Twenty-four hours after infection, transgene activation was measured via northern blot analysis (using a radiolabeled human ₂AR cDNA probe) and Western blotting (using an anti-human β₂AR antibody and horseradish peroxidase immunodetection method)(FIG. 1). A549 cells infected with 50 pfu/cell of adβ₂AR had marked increases in β₂AR mRNA and protein. Similarly treated mouse fibroblasts (3T3), a heterologous cell system, were infected with 50 pfu/cell to test for increased β₂AR expression in whole cells (FIG. 2). Adenoviral-mediated gene transfer caused significant increases in immunoreactive β₂AR expression. To test for transgene function A549 cells were used for measurement of active Na⁺ transport following infection with 50 pfu/cell of adβ₂AR for 48 hours. Following 18 hours of serum deprivation and treatment with a β₂AR specific agonist (procaterol, 10⁻⁶ M) for 10 minutes these cells had up to a 10-fold increase in cAMP levels measured in the presence of IBMX (a phosphodiesterase inhibitor), to a level similar to that seen following treatment with the adenylate cyclase activator, forskolin (10⁻⁶ M) (FIG. 3). Pretreatment with a β₂AR specific antagonist (ICI 118,551, 10⁻⁶ M) completely prevented this increased measure confirming that adenoviral mediated gene transfer of a human β₂AR gene can augment β₂AR function in alveolar epithelial cells in vitro (FIG. 3).

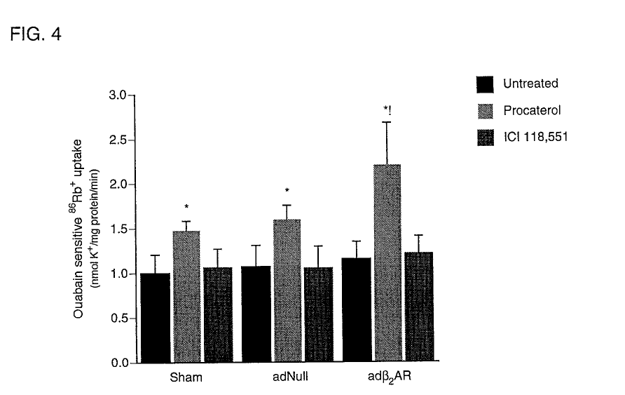

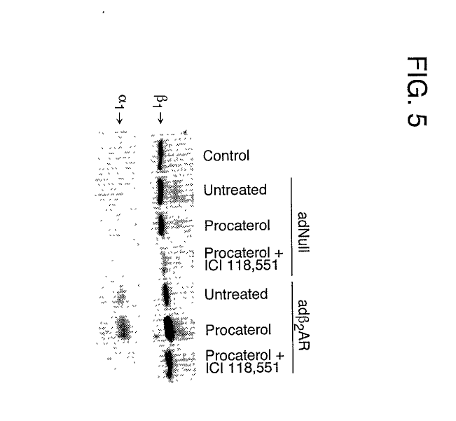

To test if β₂AR overexpression can affect active Na⁺ transport in vitro, A549 cells were treated as described above prior to measurement of ouabain-sensitive Na⁺ transport (an index of Na,K-ATPase activity). As can be seen in FIG. 4, β₂AR overexpression increased Na,K-ATPase activity by nearly 250% as compared to adNull infected controls. Additional studies done to establish the mechanisms responsible for this finding included quantification of Na,K-ATPase α₁ and β₁ expression in the basolateral membrane of A549 cells. Treatment with the β₂AR specific agonist procaterol (10⁻⁶ M) for 10 minutes caused greater increases in Na,K-ATPase expression in adβ₂AR infected cells than in adNull infected controls (FIG. 5). This recruitment of Na,K-ATPase to the cell membrane was blocked by pre-treatment with the β₂AR antagonist ICI 118,551 (10⁻⁶ M×10 minutes prior to addition of procaterol). This data indicates that receptor overexpression increases active Na⁺ transport, in part, by increasing the number of Na,K-ATPases in the cell membrane.

### Example 2

### Adenoviral-mediated gene transfer to rat lungs in vivo.

To test if β₂AR gene transfer could increase transgene expression and if overexpression could affect the transepithelial osmotic gradient necessary to achieve pulmonary edema clearance, normal Sprague-Dawley rats were infected with the vectors disclosed in Example 1. 250-300 gm male Sprague-Dawley rats were lightly sedated with pentobarbital prior to orotracheal intubation with a 14 g angiocatheter. Adenovirus (4×10⁹ pfu) was suspended in 800 ml of a 50% surfactant/50% dialysis buffer mixture (Surfanta, Abbott Laboratories, Columbus, Ohio). Immediately prior to instillation of 200 μl of adenovirus solution, the thorax of each rat was circumferentially squeezed to approach residual lung volume and to stimulate the rat to exhale. Four instillations, at five minute intervals interspersed with 90° rotations of the animal, were used facilitate uniform delivery of the adenovirus. Immediately following endotracheal instillation of adenovirus/vehicle, compression of the rat was relinquished allowing the animal to take a deep, forceful inspiration that facilitated widespread, distal dispersion of adenoviral vector. This method has been previously shown to be capable of achieving widespread viral delivery needed for studies of adenoviral-mediated β₂AR overexpression in vivo.

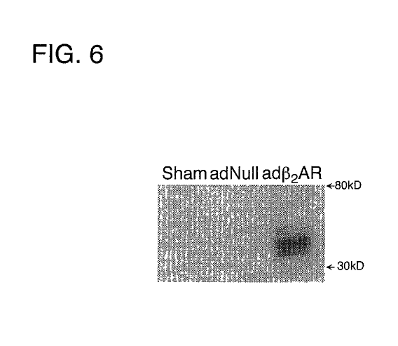

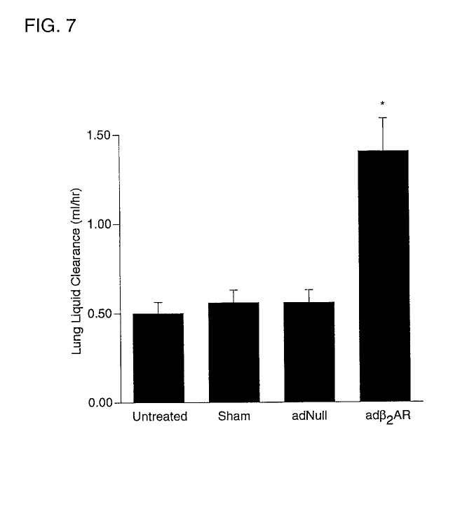

Seven days after infection, whole lung transgene expression was measured via Western analysis using a rabbit anti-human β₂AR antibody and chemiluminescent detection method. Sham and adNull infected controls had no detectable human β₂AR expression, whereas adβ₂AR infected lungs had significant levels of immunoreactive human β₂AR protein (FIG. 6). To test for in vivo transgene function lung liquid clearance was measured using a well-established isolated lung preparation. This method includes instillation of an isotonic, iso-osmotic solution that contains ²²Na⁺, ³H-mannitol and Evan's Blue Tagged albumin into the airspace. The vasculature is perfused at constant pressure with a similar isotonic solution that contains FITC-tagged albumin. The lungs are immersed in a bath consisting of an isotonic, buffered salt solution where temperature and pH are maintained within normal physiologic ranges. Changes in concentration of alveolar Evan's Blue Albumin over a sixty minute experimental period are used to calculate lung liquid clearance. Movement of labeled substances between airspace and vascular compartments is used to measure alveolar and endothelial permeability (Azzam et al., 1999; Barnard et al., 1997; Factor, et al., 1998a; Olivera et al., 1995; Olivera et al., 1994; Rutschman et al., 1993; Saldias, et al., 1998; Sznajder et al., 1995). As compared to controls, adβ₂AR increased lung liquid clearance by >150% (FIG. 7). These results indicate that overexpression of a β₂AR gene can augment β₂AR expression and function in vivo.

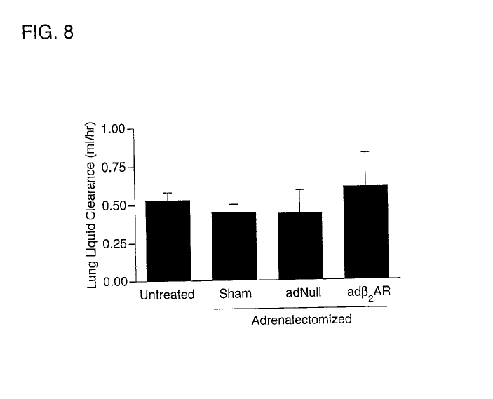

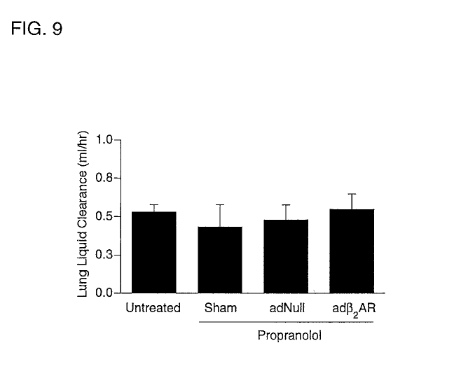

To ascertain the contribution of endogenous catecholamines to the increase in clearance noted following β₂AR gene transfer to rat lungs, 2 additional groups of rats were infected with adβ₂AR. First, adrenalectomized rats were studied, these animals have been reported to have decreased serum catecholamine levels. Clearance measured following infection with adβ₂AR was not different from controls (FIG. 8). The second group of animals was treated with the non-specific β-adrenergic receptor antagonist propranolol for 5 days prior to measurement of lung liquid clearance. Like the adrenalectomized rats clearance in these animals was not different from normal controls (FIG. 9). These studies of animals with reduced catecholamine levels or in whom the effects of endogenous catecholamines have been blocked suggests that the increased clearance noted in adβ₂AR infected rats is due to increased responsiveness to endogenous catecholamines.

### Example 3

### β-agonists cause physiologically significant β₂AR desensitization in lung epithelial cells and rat lungs.

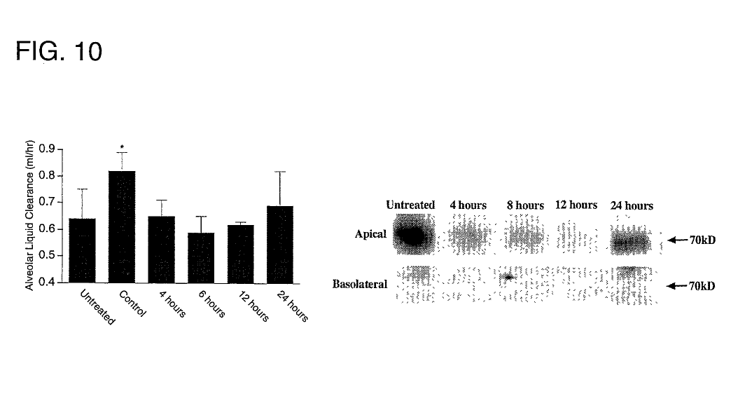

To test if agonist-induced loss of receptor function and/or expression occurs in the alveolar epithelium normal rats were treated with the β₂AR specific agonist procaterol (100 μg every 8 hours by gavage). Lung liquid clearance was then measured using a fluid-filled isolated lung preparation in the presence of procaterol (10⁻⁶ M instilled into the alveolar airspace and perfused through the pulmonary artery) beginning 4 hours after the first dose. As can be seen in FIG. 10 clearance in these animals was not different from untreated controls. Clearance increased toward normal by 24 hours but remained less than naive rats treated with procaterol only during measurement of clearance. To test if β-agonist treatment caused receptor downregulation apical membranes were isolated from peripheral lung tissue. Western analysis of these membranes revealed marked reductions in β₂AR expression indicating that proloned treatment with a β₂AR specific agonist causes receptor downregulation. Thus, the loss of catecholamine responsive lung liquid clearance in these rats is likely due, in part, to receptor downregulation.

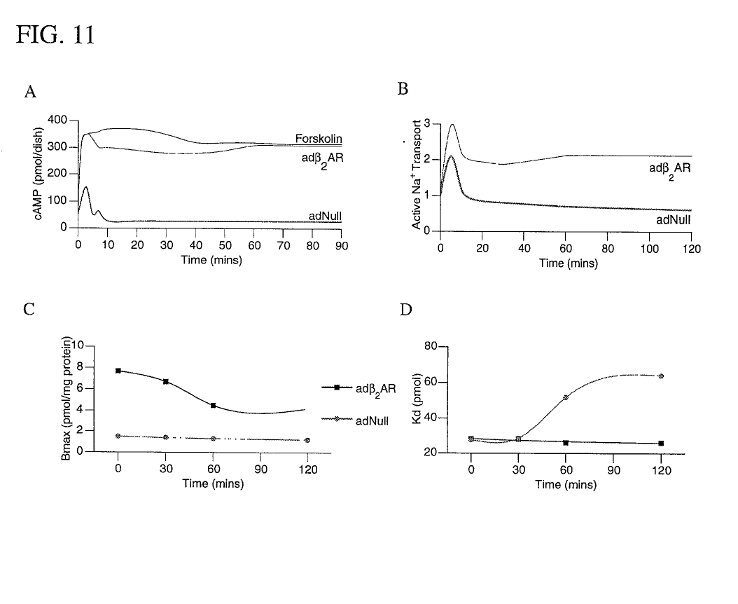

To ascertain the mechanisms responsible for this finding A549 cells were infected with adβ₂AR or adNull for 48 hours and treated with procaterol for up to 120 minutes prior measurement of receptor function (cAMP) and active Na⁺ transport. Cyclic-AMP levels, measured using a commercially available radioimmunoassay in the presence of IBMX, in adβ₂AR infected cells rose to a level similar to that seen in forskolin treated controls within 3 minutes and declined modestly but remained >10 fold above that seen in adNull infected controls (FIG. 11, graph A). Active Na⁺ transport demonstrated a similar pattern in adβ₂AR infected cells whereas transport declined by ˜30% in adNull controls (FIG. 11, graph B). This data indicates that receptor overexpression can attenuate agonist-induced receptor desensitization in A549 cells. Additional investigations included measurement of ¹²⁵I-CYP binding to membrane fractions isolated from similarly treated A549 cells. As can be seen in FIG. 11, Graph C, receptor number in adβ₂AR infected A549 cells was increased by 500% prior to treatment with procaterol and declined by 50% with procaterol treatment, but receptor number remained significantly greater than in adNull controls. Skatchard analysis of ¹²⁵I-CYP binding indicated that receptor affinity (K_{d}, an index of receptor phosphorylation) remained normal in adβ₂AR infected cells but declined with time in adNull infected controls suggesting that overexpression overwhelms endogenous regulatory mechanisms that allow for sustained membrane levels and function of the β₂AR despite treatment with a β-agonist (FIG. 11, graph D). These findings support the finding that adenoviral-mediated gene transfer of a β₂AR represents a novel application of gene therapy that functions by overcoming regulatory processes that may contribute to the pathophysiology of pulmonary edema (e.g. loss of responsiveness to endogenous catecholamines). These studies represent the first data to show that β₂AR function can be improved in the lung.

### Example 4

### β₂AR gene transfer using a high-capacity, helpervirus-dependent adenovirus that encodes a human β₂AR gene.

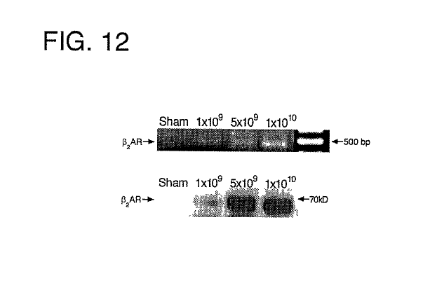

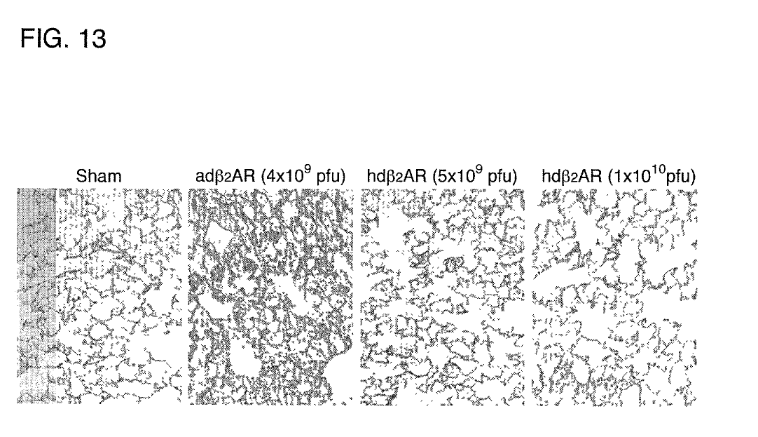

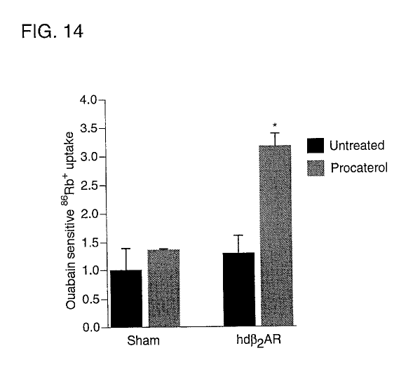

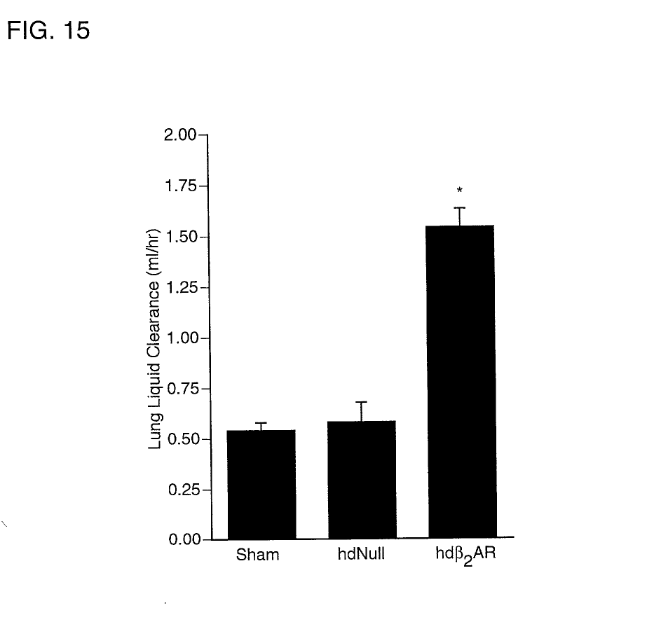

To test if a high-capacity, helper-virus dependent adenovector can affect β₂AR expression in vivo adult male-sprague-Dawley rats were infected with 1×10⁹ to 1×10¹⁰ pfu a high-capacity, helpervirus-dependent adenovirus that expresses a human β₂AR cDNA (hdβ₂AR) for 72 hours prior to measurement of transgene expression. rtPCR of total RNA harvested from peripheral lung tissue revealed significant levels of a human β₂AR mRNA that was not noted in sham controls (FIG. 12). Similarly, Western analysis of protein from peripheral lung demonstrated the presence a human β₂AR protein only in hdβ₂AR infected lungs (FIG. 12). Histologic analysis of these lungs showed no increase in cellularity or thickening of alveolar walls, these lungs were indistinguishable from sham infected controls whereas lungs infected with adβ₂AR (an otherwise identical 1^{st} generation adenovirus) caused significant histologic injury (FIG. 13). Prior studies using these doses of 1^{st} generation adenovectors caused significant toxicity and high mortality rates in rats. This data represents the first use of high-capacity, helpervirus-dependent adenovirus mediated gene transfer to lung. To test if hdβ₂AR had functional affects, A549 cells were infected with 50 pfu/cell for 48 hours prior to measurement of catecholamine responsive active Na⁺ transport (i.e. ouabain-sensitive ⁸⁶Rb⁺ uptake in the presence of 10⁻⁶ M procaterol). Active transport in hdβ₂AR infected cells was increased by >300% indicating that like the 1^{st} generation vector adβ₂AR, hdβ₂AR could positively affect catecholamine responsive active Na⁺ transport (FIG. 14). To test if hdβ₂AR could affect active Na⁺ transport in vivo, the lungs of Sprague-Dawley rats were infected with 1×10¹⁰ pfu of hdβ₂AR for 72 hours prior to measurement of lung liquid clearance using a fluid-filled isolated lung preparation. As can be seen in FIG. 15, clearance in these animals was increased by 300% as compared to sham and hdNull infected controls, without catecholamine supplementation. Unlike past experiments using 1^{st} generation vectors at this time-point post-infection, alveolar permeability for large and small solutes was not different from uninfected or sham infected controls suggesting that hdβ₂AR does not injure the alveolar epithelium. To date there are no reports of the use of similar viral vectors for lung gene transfer, nor are there any similar non-injurious vectors available for lung gene therapy, as such these findings represent novel and important advances in the field of lung gene therapy.

### MATERIALS AND METHODS

### Production of helper Virus Dependent Adenoviral Vectors

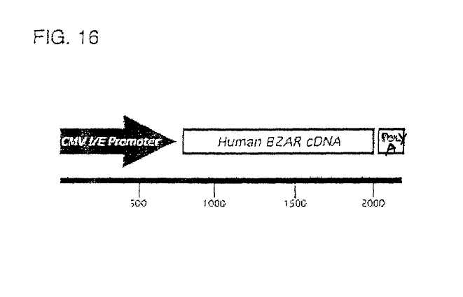

The invention described herein employs the use of helper-virus dependent adenoviral vectors that are devoid of all adenoviral protein encoding genes. These vectors are produced by transfection of a helper-dependent plasmid vector that contains a human β₂AR containing expression cassette (FIG. 16) into HEK293cre4 cells followed by infection with a replication deficient adenovirus that provides adenoviral protein sequences in trans position thereby allowing the generation of recombinant adenoviruses containing a genome comprised of DNA sequences from pHDV (vide infra). The packing sequences of the helper-virus are flanked by loxP cites, when transfected into cells that expresses Cre recombinase (e.g. HEK293cre4) the sequences between the loxP sites (i.e. the packaging signal) is excised rendering it unpackagable. This virus also contains a firefly lucifierase gene that allows ready detection of contamination by this adenovirus in subsequent steps of helper-virus dependent adenovirus production and use.

1. Production of pCMβ₂AR

A human β₂AR cDNA was obtained using the polymerase chain reaction using primers produced from sequence data from Genbank. This DNA fragment was inserted into the multiple cloning site of pcDNA 3.1 (Stratagene) following elimination of 3′ and 5′ Pme 1 sites using standard DNA cloning methods (Maniatis et al., 1989). The shuttle plasmid vector thus generated (designated pCMVβ₂AR) includes an expression cassette containing a human cytomegalovirus immediate-early promoter element, a β₂AR cDNA and a SV40 t intron polyadenylation signal.

2. Production of pHVβ₂AR

A helper dependent plasmid disclosed employs a eukaryotic expression phagemid backbone that includes an E. coli origin of replication and an ampicillin resistance gene (e.g., pBluescript SK+ (Stratagene). Inserted into the polycloning site was an ˜10 kb helper-dependent vector into which an expression cassette can be inserted. The 5′ end of the helper dependent vector consists of a human adenovirus type 5 inverted terminal repeat and packaging signal from a wild-type adenoviral genome. The 3′ end contains a 3′ inverted terminal repeat from a wild-type adenoviral genome. The 10 kb EcoR1 digestion fragment of intronic human HGPRT DNA is inserted in between the inverted terminal repeats to produce pHV10. This plasmid is propagated in recombinase negative E. coli (e.g. Sure2 cells, Clontech).

3. Production of hdβ₂AR

The helper-dependent vector portion of pHVβ₂AR is excised from its backbones via restriction endonuclease digestion and isolated via agarose gel electrophoresis, followed by transfection, using a lipid based methodology (Lipofectin, Gibco-BRL, Bethesda, Md.) into HEK293cre4 cells. These cells are stable transformants that express both adenovirus E1a and cre recombinase genes (Morral, et al., 1998; Morsy, et al., 1998; Parks, et al., 1996; Parks and Graham, 1997). Approximately 18-24 hours following transfection these cells are infected with 1 pfu/cell of a packaging incompetent, E1a⁻ adenovirus, adLC8cluc. An alternate method uses simultaneous calcium phosphate co-precipitation of DNA fragments with adLC8cluc. The packaging sequence of adLC8cluc is flanked by IoxP sites. When expressed in Cre expressing cells DNA sequences in between the IoxP sites are excised. The removal of packaging sequences from adLC8cluc renders it unable to be inserted into the adenoviral capsid (e.g. unpackageable). The remainder of the adLC8cluc genome provides adenoviral protein genes in trans that produce adenoviral capsids into which the helper-dependent, packaging competent DNA from pCMV dependent vectors is below that expected to be capable of packaging (Parks and Graham, 1997). Spontaneous combination (concatamerization) of 2 helper-dependent vectors into a single genome that is between 75% and 105% of the wild-type adenoviral genome size has been shown to occur spontaneously in HEK293cre4 cells making the method effective for the production of helper-dependent adenoviral vectors that are devoid of wild-type adenoviral protein encoding sequences (Morsy, et al., 1998). pHV10β₂AR was carefully designed to result in the production of an adenoviral genome that does not exceed 31 kb following concatamerization. The masses of these vectors are substantially less than the genome of adLC8cluc (35.3 kb)(Parks, et al., 1996). This allows efficient separation of helper-dependent adenoviruses from unexpected contamination/carryover of adLC8cluc. The β₂AR containing helper-virus dependent adenoviruses thus produced is hdβ₂AR.

4. Propagation of hdβ₂AR

Twenty-four to 48 hours after transfection and infection, HEK293cre4 cells are harvested and thermally disrupted by 6 cycles of freezing and thawing. The resultant crude viral lysate is cleared of cellular debris by low speed centrifugation and used to infect 5-10, 15 cm plates of HEK293cre4 cells that are simultaneously infected with 1-3 pfu/cell of adLC8cluc. Cells are again harvested after 24 hours and thermally disrupted followed by CsCl density gradient ultracentrifugation and dialysis to remove residual CsCl. Purified virus is then used to co-infect larger quantities (20-30, 15 cm plates) of HEK293cre4 cells. CsCl is removed by dialysis against phosphate buffered saline containing 10% glycerol. Helper-dependent vector titer is quantified by optical density readings at 260 nm (1OD=10¹² viral particles) and via plaque production counts in HEK293cre4 cells previously infected with adLC8cluc. The presence of adLC8cluc is assayed via plaque production counts following infection of HEK293 cells and by measurement of luminescence in cell lysates using a luminometer. The presence of wild-type adenovirus is assayed by measurement of plaque production counts following infection of human A549 cells and via PCR using E1a specific oligonucleotide primers.

### DOCUMENTS CITED

Azzam, Z., Saldias, F., Ridge, K., and Sznajder, J., Am J Respir Crit Care Med. 1999, 159: A603.

Barnard, M. L., Olivera, W. G., Rutschman, D. M., Bertorello, A. M., Katz, A. I., and Sznajder, J. I., Am J Respir Crit Care Med. 1997, 156: 709-14.

Berthiaume, Y., Broaddus, V. C., Gropper, M. A., Tanita, T., and Matthay, M. A., J Appl Physiol. 1988, 65: 585-93.

Berthiaume, Y., Staub, N. C., and Matthay, M. A., J Clin Invest. 1987, 79: 335-43.

Bertorello, A. M., Ridge, K. M., Chibalin, A. V., Katz, A. I., and Sznajder, J. I., Am J Physiol. 1999, 276: L20-7.

Brody, S., and Crystal, R., Ann. N.Y. Acad. Sci. 1994, 716: 90-101.

Brody, S. L., Metzger, M., Canel, C., Rosenfeld, M., and Crystal, R., Hum. Gene Ther. 1994, 5: 821-836.

Campbell, A. R., Folkesson, H. G., Berthiaume, Y., Gutkowska, J., Suzuki, S., and Matthay, M. A., J Appl Physiol. 1999, 86: 139-51.

Carstairs, J. R., Nimmo, A. J., and Barnes, P. J., Am Rev Respir Dis. 1985, 132: 541-7.

Consortium, The Large State Peer Review Organization, Arch Int Med. 1997, 157: 1103-1108.

Effros, R. M., Mason, G. R., Hukkanen, J., and Silverman, P., J. Appl. Physiol. 1989, 66: 906-919.

Factor, P., Saldias, F., Ridge, K., Dumasius, V., Jaffe, H. A., Barnard, M., Mercer, R., Perrin, R., Blanco, G., and Sznajder, J. I., J Clin Invest. 1998a, 102: 1142-14.

Factor, P., Senne, C., Dumasius, V., Ridge, K., Ari Jaffe, H., Uhal, B., Gao, Z., and Iasha Sznajder, J., Am J Respir Cell Mol Biol. 1998b, 18: 741-9.

Goodman, B. E., Brown, S. E., and Crandall, E. D., J Appl Physiol. 1984, 57: 703-10.

Goodman, B. E., Fleischer, R. S., and Crandall, E. D., Am J Physiol. 1983, 245: C78-83.

Hall, J. B., and Wood, L. D. H. Pulmonary edema. In: Current Therapy in Respiratory Medicine, Cherniack R, (ed), Series,. B. C. Dekker, Toronto, Canada, 1986, pp. 222-227.

Icard, P., and Saumon, G., Am J Physiol. 1999, 277: L1232-8.

Katkin, J., Husser, R., Langston, C., and Welty, S., Hum. Gene Ther. 1997, 8: 171-6.

Lane, S. M., Maender, K. C., Awender, N. E., and Maron, M. B., Am J Respir Crit Care Med. 1998, 158: 760-8.

Lasnier, J. M., Wangensteen, O. D., Schmitz, L. S., Gross, C. R., and Ingbar, D. H., J Appl Physiol. 1996, 81: 1723-9.

Liggett, S. B., Am. J. Respir. Cell Mol. Biol. 1997, 156: S156-S162.

Maniatis, T., Frisch, E., and Sambrook, J.: Molecular cloning: A laboratory manual, (1989) (ed 2). Cold Spring Harbor, N.Y., Cold Spring Harbor Laboratory

Martinez, F. D., Graves, P. E., Baldini, M., Solomon, S., and Erickson, R., J Clin Invest. 1997, 100: 3184-8.

Matthay, M. A., and Wiener-Kronish, J. P., Am Rev Respir Dis. 1990, 142: 1250-7.

McGrory, W., Bautista, D., and Graham, F., Virology. 1988, 163: 614-617.

Minakata, Y., Suzuki, S., Grygorczyk, C., Dagenais, A., and Berthiaume, Y., Am J Physiol. 1998, 275: L414-22.

Modelska, K., Matthay, M., Brown, L., Deutch, E., Lu, L., and Pittet, J., Am J Physiol Lung Cell Mol Physiol. 1999, 276: L844-L857.

Morral, N., O'Neal, W., Rice, K., Leland, M., Kaplan, J., Piedra, P. A., Zhou, H., Parks, R. J., Velji, R., Aguilar-Cordova, E., Wadsworth, S., Graham, F. L., Kochanek, S., Carey, K. D., and Beaudet, A. L., Proc Natl Acad Sci USA. 1999, 96: 12816-21.

Morral, N., Parks, R. J., Zhou, H., Langston, C., Schiedner, G., Quinones, J., Graham, F. L., Kochanek, S., and Beaudet, A. L., Hum Gene Ther. 1998, 9: 2709-16.

Morsy, M., Gu, M., Motzel, S., Zhao, J., Lin, J., Su, Q., Allen, H., Franlin, L., Parks, R., Graham, F., Kochanek, S., Bett, A., and Caskey, C., Proc. Natl. Acad. Sci. 1998, 95: 7866-7871.

Nishikawa, M., Mak, J. C., Shirasaki, H., and Barnes, P. J., Eur J Pharmacol. 1993, 247: 131-8.

Nishikawa, M., Mak, J. C., Shirasaki, H., Harding, S. E., and Barnes, P. J., Am J Respir Cell Mol Biol. 1994, 10: 91-9.

Olivera, W., Ridge, K., and Sznajder, J., Am J Respir Crit Care Med. 1995, 152: 1229-34.

Olivera, W., Ridge, K., Wood, L. D., and Sznajder, J. I., Am J Physiol. 1994, 266: L577-84.

Parks, R. J., Chen, L., Anton, M., Sankar, U., Rudnicki, M. A., and Graham, F. L., Proc Natl Acad Sci USA. 1996, 93: 13565-70.

Parks, R. J., and Graham, F. L., J. Virol. 1997, 71: 3293-3298.

Pittet, J. F., Wiener-Kronish, J. P., McElroy, M. C., Folkesson, H. G., and Matthay, M. A., J Clin Invest. 1994, 94: 663-71.

Rutschman, D., Olivera, W., and Sznajder, J., J. Appl. Physiol. 1993, 75: 1574-1580.

Sakuma, T., Folkesson, H. G., Suzuki, S., Okaniwa, G., Fujimura, S., and Matthay, M. A., Am. J. Resp. Crit. Care Med. 1997, 155: 506-512.

Saldias, F., Lecuona, E., Friedman, E., Barnard, M. L., Ridge, K. M., and Sznajder, J. I., Am J Physiol. 1998, 274: L694-701.

Saldias, F. J., Comellas, A., Ridge, K. M., Lecuona, E., and Sznajder, J. I., J Appl Physiol. 1999, 87: 30-5.

Staub, N. C., Physiol Rev. 1974, 54: 678-811.

Stout, J. T., and Caskey, C. T., Annu Rev Genet. 1985, 19: 127-48.

Sznajder, J. I., Olivera, W. G., Ridge, K. M., and Rutschman, D. H., Am J Respir Crit Care Med. 1995, 151: 1519-25.

Sznajder, J. I., Zucker, A., Wood, L. D. H., and Long, G. R., Am. Rev. Respir. Dis. 1986, 34: 222-228.

Tibayan, F. A., Chesnutt, A. N., Folkesson, H. G., Eandi, J., and Matthay, M. A., Am J Respir Crit Care Med. 1997, 156: 438-44.

van Ginkel, F. W., McGhee, J. R., Liu, C., Simecka, J. W., Yamamoto, M., Frizzell, R. A., Sorscher, E. J., Kiyono, H., and Pascual, D. W., J Immunol. 1997, 159: 685-93.

Walters, R., Grunst, T., Bergelson, J., Finberg, R., Welsh, M., and Zabner, J., J. Biol. Chem. 1999, 274: 10219-10226.

I claim:

1. A method for reducing pulmonary edema in acquired diseases of the mammalian lung, said method comprising:
(a) obtaining a recombinant genetic vector comprising
(i) an adenovirus that has no nucleotide sequences encoding adenovirus proteins E1a, E1b and E3; and
(ii) nucleotide sequences encoding a human β₂AR gene at levels that are an overexpression compared to levels in lung cells not having the genetic vector; and
(b) transferring the genetic vector into epithelial cells of the lung under conditions allowing expression of the nucleotide sequence.

2. A recombinant genetic vector comprising:
(a) an adenovirus that has no nucleotide sequences encoding adenovirus proteins E1a, E1b and E3; and
(b) nucleotide sequences encoding a human β₂AR gene at levels that are an overexpression compared to levels in lung cells not having the genetic vector.

3. A pharmaceutical composition comprising the recombinant genetic vector of claim 2.

4. A host cell into which a recombinant genetic vector has been transferred, said vector comprising:
(a) an adenovirus that has no nucleotide sequences encoding adenovirus proteins E1a, E1b and E3; and
(b) nucleotide sequences encoding a human β₂AR gene at levels that are an overexpression compared to levels in lung cells not having the genetic vector.
